(19) United States
(12) Patent Application Publication
(10) Pub. No.: US 2009/0029366 A1
(43) Pub. Date: Jan. 29, 2009

(54) METHODS OF IDENTIFYING MODULATORS OF HYPERPOLARIZATION-ACTIVATED CYCLIC NUCLEOTIDE-GATED (HCN) CHANNELS

(75) Inventors: Dmytro Vasylyev, Plainsboro, NJ (US); Qiang Lu, East Brunswick, NJ (US); Mark Bowlby, Richboro, PA (US); Edward Kaftan, Pennington, NJ (US); Qin Shan, Plainsboro, NJ (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 12/057,309

(22) Filed: Mar. 27, 2008

Related U.S. Application Data
(60) Provisional application No. 60/908,581, filed on Mar. 28, 2007.

(51) Int. Cl. C12Q 1/68; C12N 5/06

(52) U.S. Cl. 435/6; 435/325

(57) ABSTRACT

Methods, including high-throughput methods, for identifying modulators of hyperpolarization-activated cyclic nucleotide-gated (HCN) channels.

### CROSS-REFERENCE TO RELATED APPLICATIONS

Priority is claimed to U.S. Provisional Patent Application No. 60/908,581, filed Mar. 28, 2007, which is incorporated by reference herein in its entirety.

### FIELD OF THE INVENTION

The present invention relates to the field of methods for assessing electrochemical signaling between biological cells. More particularly, the present invention relates to high throughput screening assays for identifying modulators of hyperpolarization-activated cyclic nucleotide-gated (HCN) channels.

### BACKGROUND OF THE INVENTION

The pacemaker current is a hyperpolariziation-activated, cation-selective, inward current that modulates the firing rate of cardiac and neuronal pacemaker cells. The pacemaker current contributes to spontaneous diastolic depolarization of sinoatrial node cells in the heart, and also mediates repetitive firing in neurons and oscillatory behavior in neuronal networks. The pacemaker current is activated upon hyperpolarization, is modulated by intracellular cyclic nucleotides, and involves a single channel conductance mediated by the Hyperpolarization-activated, Cyclic Nucleotide-gated (HCN) channels. (Chen et al. (2002) Trends Cardiovasc. Med. 12:42-54). Modulation of the pacemaker current offers potential therapies for reversal or control of aberrant pacemaker current in pathological conditions, such as bradycardia, stroke, epilepsy, and pain.

The identification of new drugs that have specific modulatory effects on HCN channels will be expedited by efficient screening assays. To meet this need, the present invention provides methods of identifying HCN channel modulators that are compatible with high-throughput formats. Also provided are methods for preparing stable cell lines expressing functional HCN channels.

### SUMMARY OF THE INVENTION

The present invention provides methods of identifying a modulator of a hyperpolarization-activated cyclic nucleotide gated (HCN) channel. A representative method of the invention includes the steps of (a) providing a cell expressing an HCN channel in a saline solution comprising a membrane potential sensitive dye; (b) contacting the cell with a test agent; (c) adding a sodium/potassium diluent to the saline solution of (a) to thereby induce hyperpolarization, wherein the diluent is optionally supplemented with the membrane potential sensitive dye; and (d) assaying attenuation or enhancement of hyperpolarization, as compared to a control level of hyperpolarization, wherein attenuation of hyperpolarization indicates that the test agent is an HCN channel antagonist, and wherein enhancement of hyperpolarization indicates that the test agent is an HCN channel agonist. Also provided are methods of selecting a cell line that expresses a hyperpolarization-activated cyclic nucleotide gated (HCN) channel, for example, by (a) providing a cell suspected to express a hyperpolarization-activated cyclic nucleotide gated (HCN) channel (e.g., a cellular clone expressing an HCN channel) in a saline solution supplemented with a membrane potential sensitive dye (MPSD); (b) adding a sodium/potassium diluent solution to the saline solution of (a) to thereby induce hyperpolarization; and (c) selecting a cell that is hyperpolarized upon addition of the sodium/potassium diluent solution. Cell lines produced by the disclosed methods are also provided.

### BRIEF DESCRIPTION OF THE DRAWINGS

FIG. 1 depicts patch-clamp (current clamp) recordings from a HEK-293 cell stably transfected with human HCN1. Arrows indicate the time point of application of the HCN channel inhibitors CsCl (Cs⁺, 1 mM) and ZD7288 (50 μM). Bars at top indicate salinity of the bath media over the indicated time. HBSS, Hank's Balanced Salt Solution; TEA, tetraethyl-ammonium.

FIG. 2 depicts representative conditions for an HCN channel assay as disclosed herein. Cells expressing an HCN channel are placed into a saline solution having sodium ions at a concentration of about 140 mM and potassium ions at a concentration of about 5 mM, such that the cells have a resting membrane potential of −30 to −40 mV. Addition of a saline with reduced concentration of sodium and potassium ions (herein after “sodium/potassium diluent” or “diluent”) to the saline solution results in a decrease in Na⁺ and K⁺ ions concentration and hyperpolarization to about −80 mV. The assay window is approximately 40 mV.

FIGS. 3A and 3B depict results of a MPSD FLIPR®⁹⁶ (96 well plate) assay performed using cells expressing an HCN1 channel. FIG. 3A depicts the whole-plate screenshot of the changes in fluorescence/membrane potential over time. FIG. 3B depicts the change in fluorescence as a function of the fold dilution of the saline solution. ΔF, change in fluorescence.

FIGS. 4A-4C depict the results of a MPSD FLIPR®⁹⁶ (96 well plate) assay performed using cells expressing an HCN1 channel and an HCN channel inhibitor added to the saline solution, as described in Example 5. FIG. 4A depicts the change in fluorescence (F) over time for control cells (lower trace) and for cells exposed to the HCN channel inhibitor ZD7288 at a concentration of 30 μM (upper trace). FIG. 4B depicts the whole-plate screenshot of the changes in fluorescence/membrane potential over time. Presence of the HCN channel inhibitor effectively blocked hyperpolarization. FIG. 4C shows that the Z-factor was between 0.25 and 0.75 over the course of the assay.

FIGS. 5A-5B depict the results of a MPSD FLIPR®³⁸⁴ (384 well plate) assay performed using cells expressing an HCN1 channel and an HCN channel inhibitor added to the saline solution, as described in Example 5. FIG. 5A depicts the whole-plate screenshot of the changes in fluorescence/membrane potential over time. FIG. 5B depicts the change in fluorescence over time for control cells (overlaid lower traces ) and for cells exposed to the HCN channel inhibitor ZD7288 at a concentration of 50 μM (overlaid upper traces ). Presence of the HCN channel inhibitor effectively blocked hyperpolarization. At 180 seconds, the average Z′ factor was 0.74.

FIGS. 6A and 6B depict results of a MPSD FLEX⁹⁶ (96 well plate) assay performed using cells expressing an HCN3 channel and an HCN channel inhibitor added to the saline solution, as described in Example 5. FIG. 6A depicts the whole-plate screenshot of the changes in fluorescence/membrane potential over time. FIG. 6B shows that the Z-factor was between about 0.2 and 0.7 over the course of the assay.

FIGS. 7A-7B depicts the results of a MPSD FLIPR®⁹⁶ assay using cells expressing an HCN3 channel and an HCN channel inhibitor added to the saline solution, as described in Example 5. FIG. 7A depicts the change in fluorescence (F) over time for control cells (lower trace) and for cells exposed to the HCN channel inhibitor ZD7288 (upper trace). FIG. 7B shows that the Z-factor was between 0 and 0.75 at the time of hyperpolarization.

FIG. 8 depicts the results of a MPSD FLIPR®³⁸⁴ assay performed using cells expressing an HCN4 channel and an HCN channel inhibitor added to the saline solution, as described in Example 6. FIG. 8 depicts the change in fluorescence over time for control cells (overlaid lower traces) and for cells exposed to the HCN channel inhibitor ZD7288 (overlaid upper traces). Presence of the HCN channel inhibitor effectively blocked hyperpolarization.

FIGS. 9A-9B depict the results of a MPSD FLIPR®³⁸⁴ assay performed with the addition of dimethyl sulfoxide (DMSO) to the saline solution. FIG. 9A shows a whole-plate screenshot of the change in fluorescence at the indicated concentrations (%) of DMSO. FIG. 9B quantifies the change in fluorescence (ΔF, from FIG. 9A) at the indicated concentrations (%) of DMSO.

FIGS. 10A-10B depict a dose-response analysis of blockade of hyperpolarization by the HCN channel inhibitor ZD7288. FIG. 10A shows the change in fluorescence as a function of ZD7288 concentration for a single representative experiment. FIG. 10B shows ZD7288 IC₅₀ on HCN1 for 20 representative concentration-response curves.

FIGS. 11A-11B depict the results of a MPSD FLEX⁹⁶ assay for selection of a cell line that expresses a functional HCN3 channel, as described in Example 9. FIG. 11A shows the percentage change in fluorescence (% Baseline) for representative clones. FIG. 11B shows the percentage change in fluorescence (% Baseline) for representative clones incubated in saline (Control—below baseline) or in saline plus HCN channel inhibitor ZD7288 (+ZD—above baseline).

FIG. 12 depicts the results of a MPSD FLEX⁹⁶ assay for selection of a cell line that expresses a functional HCN1 channel, as described in Example 9. The percentage change in fluorescence (% Baseline) is shown for representative clones incubated in saline (Hanks—below baseline) or in saline plus HCN channel inhibitor (ZD7288—above baseline).

FIG. 13 depicts voltage-clamp recordings of human HCN1 currents from a HEK-293H cell stably expressing a human HCN1 channel. The holding potential was −40 mV. Currents were elicited every 30 seconds by hyperpolarizing pulses from a holding voltage of −100 mV followed by a step to −70 mV.

FIGS. 14A-14D depict the results of MPSD FLIPR®³⁸⁴ assays performed using cells expressing an HCN2 channel incubated in saline with or without addition of an HCN channel inhibitor, as described in Example 10. FIGS. 14A-14D each depict the change in fluorescence over time for control cells (overlaid lower traces) and for cells exposed to the HCN channel inhibitor ZD7288 (overlaid upper traces) for the clones #4, #8, #11, and #12, respectively. Presence of the HCN channel inhibitor effectively blocked hyperpolarization.

FIG. 15 depicts the results of a focus compound library screen on HCN1 channel using MPSD FLEX³⁸⁴ assay as described in Example 5. The plate format was 88 compounds per plate; compounds were plated in quadruplicate at 30 μM. FIG. 15A depicts a whole-plate screenshot of the changes in fluorescence over time in control wells (columns 23-24) and wells containing the test compounds (columns 1-22). Three hits were identified (shaded areas). Hits were identified according to the equation ΔF_{Hit}≦MEAN+3XS.D., where MEAN is the average of RFU values in control wells and 3XS.D. is three standard deviations of RFU values in control wells. ΔF_{Control}=−17.5±2.0 (MEAN±S.D., n=32); ΔF_{Hit}≦11.5 (FIG. 15B).Functional activity of these hits on HCN1 channel was confirmed by whole-cell voltage-clamp recordings.

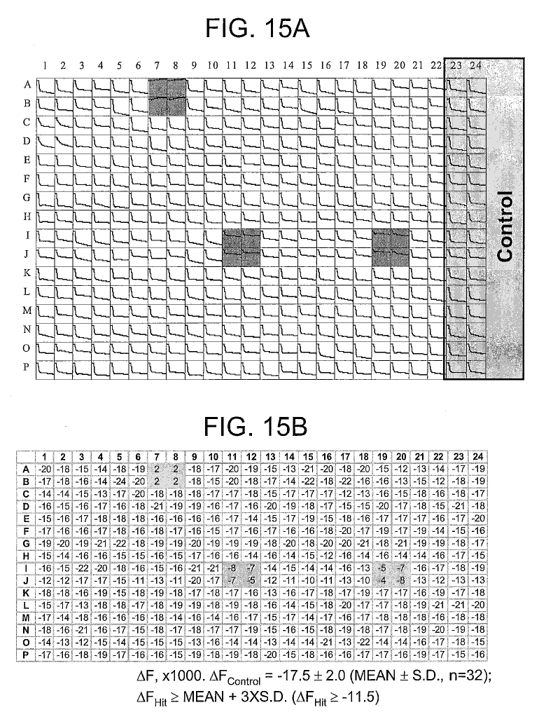

### DETAILED DESCRIPTION OF THE INVENTION

The present invention provides methods, including high throughput methods, for identifying modulators of hyperpolarization-activated nucleotide gated (HCN) channels. Also provided are methods for preparing stable cell lines expressing HCN channels.

### I. HCN Channels

HCN channels share structural features with voltage-gated potassium channels, including six transmembrane helices (S1-S6), a positively charged voltage sensing S4 segment, and an ion-conducting pore between S5 and S6. (Ludwig et al. (1998) Nature 393:587-591). In the C-terminal region, the channels carry a cyclic nucleotide-binding domain (CNBD).

In vertebrates, the HCN gene family includes four members, HCN1, HCN2, HCN3 and HCN4 and any of these or other isoforms and their combinations are contemplated for use with the present methods. In native tissues the four channel isoforms show differential expression resulting in different biophysical properties of native pacemaker channels. For example, HCN isoforms in the brain show different regional distribution and are expressed at different levels. HCN1, HCN2, and HCN3 proteins are widely expressed in the brain and spinal cord, while HCN4 protein expression in neural tissues is generally low. HCN2 and HCN4 proteins are also strongly expressed in the heart. In heterologous systems, HCN1 is the fastest channel (activation in the tens of milliseconds), while HCN2 and HCN3 channels activate more slowly (in hundreds of milliseconds). The HCN4 channel requires seconds to activate and is therefore the slowest channel. The different homomeric HCN channels also differ in changes in activation kinetics and steady state activation in response to cAMP. For example, cAMP causes a greater positive shift for the mid-point range of activation (V_{1/2}) for HCN2 in comparison to the shift for HCN1. HCN channels are assembled as tetramers of HCN alpha subunits (i.e., HCN1, HCN2, HCN3, and HCN4), which may be heteromers or homomers. Accordingly, another source for the variation in activation kinetics and voltage dependence of HCN channels is likely due to the coassembly of different HCN isoforms to form heteromers. See e.g., Ulens et al. (2002) J. Biol. Chem. 276:6069-6072.

Heteromeric HCN channels as well as homomeric HCN channels may be used with the present methods. These HCN alpha subunits include HCN1, HCN2, HCN3 and HCN4. A homomeric HCN channel refers to an HCN channel composed of identical alpha subunits whereas a heteromeric HCN channel refers to an HCN channel composed of two or more different types of alpha subunits.

Both homomeric and heteromeric channels may include auxiliary beta subunits. A beta subunit is a polypeptide monomer that is an auxiliary subunit of an HCN channel composed of alpha subunits; however, beta subunits alone may not form a channel (see e.g., U.S. Pat. No. 5,776,734). Beta subunits may increase the number of functional HCN channels by helping the alpha subunits reach the cell surface, changing channel activation kinetics, and/or changing the specificity and/or affinity of proteins that interact with an HCN channel. Beta subunits may be outside of the pore region of the channel, associated with alpha subunits comprising the pore region, or a component of the pore region. Optionally, the beta subunits are encoded by genes belonging to the KCNE family, such as MiRP1 and MiRP2 genes.

HCN channels are hyperpolarization-activated, cyclic nucleotide-gated membrane channels. HCN channels conduct both Na⁺ (inward flux from the extracellular milieu to the cytosol) and K⁺ (outward flux), and have a reversal potential of about −20 to −40 mV under physiological conditions.

The reversal potential for HCN channels is determined by the reversal potentials of the permeant sodium and potassium. E_{K}, or the reversal potential for potassium, is the membrane potential at which there is no net flow of potassium ion because the electrical potential (i.e., membrane potential) driving potassium influx is balanced by the concentration gradient directing potassium efflux. E_{K} depends on the concentration of potassium ions inside and outside the cell and is typically between −60 and −100 mV for mammalian cells. Similarly, E_{Na}, or the reversal potential for sodium, depends on the relative concentration of sodium found inside and outside the cell and is typically near 50 mV. Because HCN channels conduct both sodium and potassium, their reversal potential lies between E_{K} and E_{Na}, and is typically −20 to −40 mV under physiological ion concentrations. HCN channels have a greater probability of being open at membrane potentials more negative than the resting membrane potential, i.e., HCN channels have an increased probability of opening at more hyperpolarized potentials. See e.g., Luthi & McCormick, Neuron 21(1):9-12 (1998) for a general discussion of activation by hyperpolarization.

### II. Cells Expressing an HCN Channel

Suitable cells for use with the present methods include those cells which endogenously express an HCN channel, including for example, cardiac cells, such as sino-atrial node cells, atrial muscle cells, and ventricular muscle cells; neural cells such as those from the olfactory bulb, cerebral cortex, hippocampus, (e.g., hippoccampal pyramidal cells), thalamus, amygdala, superior and inferior colliculi, cerebellum (e.g., Purkinje cells), neural stem; photoreceptors, and taste buds.

Cells that heterologously express HCN channels are also suitable for use with the present methods. Nucleic acids encoding HCN channels that may be used to transform cells are well known in the art and may be found, for example, in GenBank (e.g., Accession Numbers NM_{—}005477, NM_{—}021072, NM_{—}020897 and NM_{—}001194). Such nucleic acids include those encoding a mammalian HCN channel, such as a human HCN channel, including a human HCN1 channel isoform, a human HCN2 channel isoform, a human HCN3 channel isoform, and a human HCN4 channel isoform. The nucleic acids encoding the HCN channels may encode homomeric channels or heteromeric HCN channels. Nucleic acids encoding heteromeric channels include nucleic acids encoding two or more HCN channel isoforms, for example, HCN1 and HCN2, HCN1 and HCN3, HCN1 and HCN4, HCN2 and HCN3, HCN2 and HCN4, or HCN3 and HCN4. Additional HCN channels may include three or more HCN channel isoforms.

A construct for expression of an HCN channel may include a vector and a HCN nucleotide sequence, wherein the HCN nucleotide sequence is operably linked to a promoter sequence. Representative promoters include both constitutive promoter and inducible promoters, such as Simian virus 40 early promoter, a long terminal repeat promoter from retrovirus, an actin promoter, a heat shock promoter, and a metallothien protein. A construct for recombinant HCN channel expression may also include transcription termination signals and sequences required for proper translation of the nucleotide sequence. Preparation of an expression construct, including addition of translation and termination signal sequences, is well known to one skilled in the art.

Suitable vectors that may be used to express a HCN channel include viruses such as vaccinia virus or adenovirus, baculovirus vectors, yeast vectors, bacteriophage vectors (e.g., lambda phage), plasmid vectors (e.g., pcDNA3.1, available from Invitrogen of Carlsbad, Calif.), cosmid DNA vectors, transposon-mediated transformation vectors, and derivatives thereof. Other suitable vectors include).

Constructs are introduced into a host cell using a transfection method compatible with the vector employed. Standard transfection methods include electroporation, DEAE-Dextran transfection, calcium phosphate precipitation, liposome-mediated transfection, transposon-mediated transformation, infection using a retrovirus, particle-mediated gene transfer, hyper-velocity gene transfer, and combinations thereof.

Cells into which a heterologous nucleic acid encoding an HCN channel may be introduced include eukaryotic cells such as mammalian cells (e.g., HEK-293 cells, HeLa cells, CV-1 cells, COS cells), amphibian cells (e.g., Xenopus oocytes), insect cells (e.g., Sf9 cells), as well as prokaryotic hosts such as E. coli and Bacillus subtilis.

For recombinant production of an HCN channel, cells are transfected with one or more constructs that include nucleic acids encoding an HCN channel. A single construct may encode an HCN1, HCN2, HCN3, or HCN4 homomeric channel or a heteromeric channel. Such a construct may optionally encode a beta auxiliary subunit. Alternatively, two or more constructs may be used to coexpress HCN channel alpha subunits, and optionally a beta auxillary subunit. For example, an expression vector may encode one or more subunits from HCN1 and be co-expressed with an expression vector encoding one or more HCN2 channels to form a HCN1/HCN2 heteromeric channel. Other HCN channels may be derived from the co-expression of two or more expression vectors each encoding one or more subunits of HCN1 and HCN3, HCN1 and HCN4, HCN2 and HCN3, HCN2 and HCN4, or HCN3 and HCN4. A construct encoding a beta auxiliary subunit also may be coexpressed with the one or more constructs encoding HCN channel alpha subunits.

The constructs expressing HCN channel subunits may be used to transiently or stably transfect cells. Assays of HCN hyperpolarization activity that employ transiently transfected cells may further include a marker that distinguishes transfected cells from non-transfected cells. A marker may be encoded by or otherwise associated with a construct for HCN channel expression, such that cells are simultaneously transfected with a nucleic acid molecule encoding an HCN channel subunit and the marker. Representative detectable molecules that are useful as markers include, for example, a protein product that confers resistance to a selective media, an enzyme, a fluorescent protein, a binding protein, and an antigen.

A number of selection systems may be used including the herpes simplex virus thymidine kinase (Wigler et al. (1977) Cell 11:223), hypoxanthine-guanine phosphoribosyltransferase (Szybalska & Szybalski (1992) Proc. Natl. Acad. Sci. U.S.A 48:202), and adenine phosphoribosyltransferase (Lowy et al. (1980) Cell 22:817). Also, antimetabolite resistance may be used as the basis of selection for the following genes: dhfr, which confers resistance to methotrexate (Wigler et al. (1980) Natl. Acad. Sci. U.S.A. 77:357; O'Hare et al. (1981) Proc. Natl. Acad. Sci. U.S.A. 78:1527); gpt, which confers resistance to mycophenolic acid (Mulligan & Berg (1981) Proc. Natl. Acad. Sci. U.S.A 78:2072); neo, which confers resistance to the aminoglycoside G-418 (Wu and Wu (1991) Biotherapy 3:87-95), (Mulligan (1993) Science 260:926-932), (Morgan and Anderson (1993) Ann. Rev. Biochem. 62:191-217); and hygro, which confers resistance to hygromycin (Santerre et al. (1984) Gene 30:147). Methods commonly known in the art of recombinant DNA technology may be routinely applied to select the desired recombinant clone, and such methods are described, for example, in Chapters 12 and 13, Dracopoli et al. (eds) (1994) Current Protocols in Human Genetics, John Wiley & Sons, New York and Colberre-Garapin et al. (1981) J. Mol. Biol. 150:1, which are incorporated by reference herein in their entireties.

Stably engineered cells may also be used in cell line generation. Methods for generating a stable cell line following transformation of a host cell are known in the art. See e.g., Joyner (1993) Gene Targeting: A Practical Approach, Oxford University Press, Oxford/N.Y.

The cells and cell lines which express an HCN channel as described herein may be frozen and stored for later use. Frozen cells may be readily transported for use at a remote location. Methods for preparation and handling of frozen cells may be found in Freshney (1987) Culture of Animal Cells: A Manual of Basic Technique, 2nd ed. A. R. Liss, New York and in U.S. Pat. Nos. 6,176,089; 6,140,123; 5,629,145; and 4,455,842; among other places.

### III. Methods of Identifying HCN Modulators

The present invention provides method for identifying an HCN channel modulator. The compounds thus identified may serve as lead compounds for development of therapeutic molecules or may themselves be used as therapeutic molecules. A variety of diseases may be treated by administering HCN channel modulators, e.g., conditions associated with a pacemaker current dysfunction, such as a neurological condition, cardiovascular condition, renal condition, pulmonary condition, or hepatic condition.

According to the disclosed methods, HCN channel modulators are identified by inducing hyperpolarization of HCN channel expressing cells, contacting the cells with a test agent, and observing attenuation or enhancement of the hyperpolarization as compared to control cells not contacted with the test agent. Attenuation of hyperpolarization, including a measurable reduction in the magnitude of hyperpolarization or complete blockade of hyperpolarization, indicates that the test agent is an HCN channel antagonist, i.e., an inhibitory agent that decreases, blocks, prevents, or delays activation of an HCN channel, or promotes inactivation and/or desensitization of an HCN channel. Enhancement of hyperpolarization indicates that the test agent is an HCN channel agonist, i.e., an agent that increases activation or current flow through an HCN channel. HCN antagonists and agonists may exert their modulatory effect by direct or indirect binding to an HCN channel. Modulators identified using the disclosed methods also include allosteric modulators, i.e., allosteric antagonists and agonists.

For example, the present method may include the steps of (a) providing a cell expressing an HCN channel in a physiological saline (e.g., HBSS or equivalent) solution containing a membrane potential sensitive dye; (b) contacting the cell expressing an HCN channel with a test agent, (c) adding a diluent with a low concentration of sodium and potassium ions to the saline solution of (a) to thereby induce hyperpolarization due to HCN channel activity; and (d) assaying attenuation or enhancement of hyperpolarization, as compared to a control magnitude of hyperpolarization. The diluent is optionally supplemented with the membrane potential sensitive dye so as to achieve or maintain an effect of the membrane potential sensitive dye, i.e., an amount effective to determine changes in membrane potential according to the detection method employed.

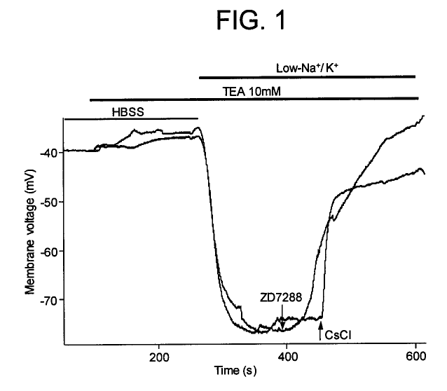

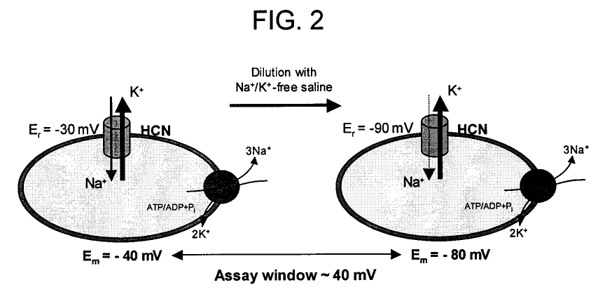

A control magnitude of hyperpolarization refers to a measurable change in membrane potential following induced hyperpolarization of a cell expressing an HCN channel under physiological conditions. As shown in FIGS. 1-2, an estimate of control magnitude of hyperpolarization is typically about −40 mV, which constitutes a representative assay window. A control magnitude of hyperpolarization may be determined in a parallel sample that includes cells not contacted with a test agent, or in the same sample of cells when the test agent is introduced simultaneously with the sodium/potassium diluent.

The step of contacting the cells expressing an HCN channel with a test agent is accomplished by adding the test agent to the saline solution at an appropriate time. For example, the test agent may be added prior to, concurrently with, or subsequent to induction of hyperpolarization, depending on the assay format that is employed. The step of contacting a cell expressing an HCN channel with a test agent also includes contacting the cell with a plurality of test agents simultaneously, such as two or more test agents, or three or more test agents, etc.

To identify an HCN channel antagonist, cells expressing an HCN channel are contacted with a test agent and inhibition of induced hyperpolarization is observed. Cells not contacted with the test agent (control cells) are assigned a relative HCN activity value of 100%, which correlates with the magnitude of induced hyperpolarization. Antagonism of HCN channels is achieved when the HCN activity value of cells contacted with the test agent is about 90% or less of the HCN activity value of control cells, for example, 80% or less, or 70% or less, or 60% or less, or 50% or less, or 40% or less, or 30% or less, or 20% or less, or 10% or less, or 1% or less. Antagonism of HCN channels is also achieved when the HCN activity value of cells contacted with the test agent is about 2-fold reduced or less as compared to the HCN activity of control cells, for example, 3-fold less, or 4-fold less, or 5-fold less, or 6-fold less, or 7-fold less, or 8-fold less, or 9-fold less, or 10-fold less, or 20-fold less, or 50-fold less, or 100-fold less, or still further reduced. Antagonism of HCN channels is also achieved when the average HCN channel activity value in cells contacted with the test agent is reduced by about 5 standard deviations (s.d. of the HCN channel activity value of control cells) from the HCN channel activity value of control cells, including, for example, a reduction of 4 standard deviations, or 3 standard deviations, or 2 standard deviations, or 1 standard deviation.

To identify an HCN channel agonist, cells expressing an HCN channel are contacted with a test agent and enhancement of induced hyperpolarization is observed. Agonism of HCN channels is achieved when the HCN activity value of cells contacted with the test agent is at least about 10% greater than to the HCN activity value of control cells, for example, at least about 20% greater, or at least about 30% greater, or at least about 40% greater, or at least about 50% greater, or at least about 60% greater, or at least about 70% greater, or at least about 80% greater, or at least about 80% greater, or at least about 100% greater, or at least about 500% greater, or at least about 1000% greater, or more. Agonism of HCN channels is also achieved when the HCN activity value of cells contacted with the test agent is about 2-fold greater as compared to the HCN activity of control cells, for example, 3-fold greater, or 4-fold greater, or 5-fold greater, or 6-fold greater, or 7-fold greater, or 8-fold greater, or 9-fold greater, or 10-fold greater, or 20-fold greater, or 50-fold greater, or 100-fold greater, or even greater.

The disclosed methods of identifying HCN channel modulators are compatible with multiple throughput and high throughput assay formats, e.g., simultaneous evaluation of 96 samples, or 384 samples, or more. Thus, the assays may be performed to assess the HCN channel modulatory activity of at least about 10³ test agents per day, or at least about 10⁴ test agents per day, or at least about 10⁵ test agents per day, or at least about 10⁶ test agents per day, or at least about 10⁷ test agents per day.

Changes in the signal intensity of a membrane potential sensitive dye may be measured using any one of appropriate devices for such purpose. The FLIPR® (Molecular Devices, Inc. of Sunnyvale, Calif.) and FLEXSTATION® devices are employed in the examples described herein.

The Z-factor (Z′) is a measure of the quality or power of a high-throughput screening (HTS) assay. The Z-factor value approaches 1.0 when the assay includes a large dynamic range with small standard deviations. A Z-factor value between 1.0 and 0.5 is considered an excellent, robust assay. See Zhang et al. (1999) J. Biomol. Screen. 4(2):67-73. As described in the examples, the Z-factor calculations for the assays of the present invention indicate that they are robust assays.

### III.A. Hyperpolarizing Conditions

The methods of the present invention are useful for identifying modulators, i.e., antagonists and agonists of HCN channels, by comparing a change in the magnitude of induced hyperpolarization in the presence and absence of a test agent. Hyperpolarizing conditions are induced by first incubating the cell in a saline solution and then diluting the concentration of sodium and potassium in the saline solution by the addition of a diluent with a reduced sodium and/or potassium ions concentration.

Any saline solution that contains a physiological concentration of potassium and sodium ions may be used. For example, the amount of sodium ions in the incubating saline solution is at least about 50 mM, such as at least about 100 mM, 120 mM, 125 mM, 130 mM, 135 mM, or 140 mM. The amount of potassium ions in the incubating saline solution is at least about 3 mM, such as at least about 5 mM, 10 mM, 50 mM, 100 mM, or 150 mM.

Representative incubating saline solutions include Ringer's Lactate (155 mM NaCl, 5 mM KCl, 2 mM CaCl₂, 1 mM MgCl₂, 2 mM NaH₂PO₄, 10 mM HEPES, and 10 mM glucose, at pH 7.2), Tyrode's buffer (137 mM NaCl, 12 mM NaHCO₃, 26 mM KCl, 5.5 mM glucose, 0.1% BSA, and 5.0 mM Hepes at pH 7.35), Kreb's buffer (119 mM NaCl, 4.7 mM KCl, 2.5 mM CaCl₂, 1.17 MgSO₄, 25 mM NaHCO₃, 1.18 mM KH₂PO₄, 0.026 mM EDTA, and 5.5 mM glucose), modified Hank's balanced salt (HBSS) (143 mM NaCl, 5.6 mM KCl, 2 mM MgCl₂, 10 mM HEPES, 10 mM glucose, 0.2 mM CaCl₂, and 0.4% BSA, at pH 7), and Hank's balanced salt solution (HBSS) (1.26 mM CaCl₂, 5.33 mM KCl, 0.44 mM KH₂PO₄, 0.41 mM MgSO₄, 137.93 mM NaCl, 4.17 mM NaHCO₃, 0.34 mM Na₂HP₄, 0.49 MgCl₂, and 5.56 mM glucose at pH 7.4).

A representative sodium/potassium diluent solution dilutes the sodium and potassium ions in the saline solution by at least about 5-fold, such as 10-fold, 20-fold, 30-fold, 50-fold, or more. Because the ionic composition of the saline solution is reduced in the presence of the sodium/potassium diluent, isotonicity may be retained by the addition of an HCN channel-impermeant cation in an amount sufficient to maintain isotonic conditions of the diluent saline solution. Isotonic conditions refer to an osmolality that is within the range tolerated by the cell or a solution that has the same osmotic pressure as the interior of the cell. Usually this is in the range of about 285-315 mOsm/kg H₂O depending on the cell type and source. Accordingly, the saline solution after addition of the sodium/potassium diluent solution may have an isotonicity between about 290-305 mOsm/kg H₂O. The impermeant cations may be added to the incubating solution and/or be contained in the sodium/potassium diluent solution. Representative impermeant cations include N-methyl-D-glucamine, choline, choline-chloride, tetraethylammonium (TEA), tetrethymethyammonium (TMA) and tetrapropylammonium (TPA).

When the incubation saline is HBSS, the sodium/potassium diluent solution decreases the amount of sodium ions in the saline solution to at least about 30 mM or less, such as 25 mM, or 20 mM, or 10 mM, or less. The sodium/potassium diluent solution also decreases the amount of potassium ions in the saline solution to less than about 10 mM, such as, less than 5 mM, or less than 2 mM, or less than 0.8 mM, or less.

### III.B. Membrane Potential Sensitive Dyes

In performing the disclosed assay methods, the saline solution and the sodium/potassium diluent solution described above may each further include a membrane potential sensitive dye to assess hyperpolarization of an HCN channel expressing cell. Generally, membrane potential sensitive dyes partition across the cytoplasmic membrane of the HCN channel expressing cells, and depend on the membrane potential across the plasma membrane. The intensity of the membrane potential sensitive dye increases when the dye is bound to cytosolic proteins. When the cells are depolarized, more dye enters the cells and the increased intracellular concentration of dye binding to intracellular lipids and proteins causes an increase in signal. When the cells are hyperpolarized, dye exits the cells and the decreased intracellular concentration of dye binding to lipids and proteins results in a decrease of signal.

Representative membrane potential sensitive dyes useful in the present invention include cationic or zwitterionic styryl dyes, cationic rhodamines, anionic oxonols, hybrid oxonols and merocyanine. See www.probes.com/handbook/sections/2300.html on-line 1999 version of the Haugland (1999) Handbook of Fluorescent Probes and Research Chemicals, Sixth Edition, Molecular Probes, Inc., Eugene, Oreg., and the references cited therein; Loew, (1994) Adv Chem Ser 235: 151; Wu and Cohen; Smith, (1990) Biochim. Biophys. Acta. 1016:1; Gross and Loew, (1989) Meth. Cell. Biol 30:193; Freedman and Novak (1989) Meth. Enzymol. 172:102; Wilson and Chused (1985) Journal of Cellular Physiology 125:72-81; Epps et. al. (1993) Chemistry of Physics and Lipids 69:137-150; and Tanner et al. (1993) Cytometry 14:59-69.

The membrane potential-sensitive dye may be a slow response dye, which can be used to measure membrane potential by virtue of voltage-dependent dye redistribution and fluorescence changes resulting from the redistribution, such as a change of the concentration of the fluorophore within the cell or vesicle, a change in the dye fluorescence due to aggregation, or a change in dye fluorescence due to binding to intracellular or intravesicular sites. Representative slow response dyes include bis-(1,3-dibutylbarbituric acid) trimethane oxonol [DiSBac₂(3)] or bis-(1,3-dibutylbarbituric acid)pentamethine oxonol [DiBac₄(5)]. Other suitable slow response dyes include the carbocyanine type dyes, such as diOC5(3)-3,3′-dipenyloxacarbocyanine iodide, diOC6(3)-3,3′-dihexloxacarbocyanine iodide, and JC-1 5,5′,6,6′-tetrachloro-1,1′-3,3′-tetraethylbenzimidazolecarbocyanine iodide, and rhodamine dyes, such as rhodamine-123.

Fast response dyes may also be used in the disclosed assay methods. Optical response of these dyes is sufficiently fast to detect transient (millisecond) potential changes in excitable cells. The magnitude of the membrane potential-dependent fluorescence change may be small, such as a 2-10% fluorescence change per 100 mV potential change. Representative fast response dyes include styrylpyridinium dyes. See e.g., Haugland, 1999, Section 23.2.

### III.C. Test Agents

A test agent refers to any agent that potentially interacts with an HCN channel, subunit or any component thereof. Test agents include any synthetic, recombinant, or natural product or composition. A test agent suspected to interact with a HCN channel, or subunit or other component thereof may be evaluated for such an interaction using the methods disclosed herein.

Representative test agents include, but are not limited to, small molecules (e.g., chemical compounds), antibodies or fragments thereof, peptides, proteins, oligopeptides (e.g., from about 5 to about 25 amino acids in length, such as from about 10 to 20 or 12 to 18 amino acids in length, such as 12, 15, or 18 amino acids in length), polysaccharides, lipids (e.g., a sphingolipid), fatty acids, nucleic acids (e.g., aptamers), nucleic acid-protein fusions, any other affinity compound, and combinations thereof. A test agent may be a purified molecule, a homogenous sample, or a mixture of molecules or compounds.

A small molecule refers to a compound, for example an organic compound, with a molecular weight of less than about 1,000 daltons, such as less than about 750 daltons, or less than about 600 daltons, or less than about 500 daltons. A small molecule may have a computed log octanol-water partition coefficient in the range of about −4 to about +14, such as in the range of about −2 to about +7.5.

Test agents also include antibodies capable of specifically binding HCN channels or subunits thereof, including polyclonal antibodies, monoclonal antibodies (mAbs), humanized or chimeric antibodies, human monoclonal antibodies, single chain antibodies, Fab fragments, F(ab′)₂ fragments, fragments produced by a Fab expression library, anti-idiotypic (anti-Id) antibodies, and epitope-binding fragments of any of the above.

It will be appreciated that there are many suppliers of chemical and biological compounds, including Sigma-Aldrich (St. Louis, Mo.), Fluka Chemika-Biochemica Analytika (Buchs Switzerland), and the like. Test agents may be obtained or prepared as a library or collection of molecules. A library may contain a few or a large number of different molecules, varying from about ten molecules to several billion molecules or more. A plurality of test agents in a library may be assayed simultaneously. Optionally, test agents derived from different libraries may be pooled for simultaneous evaluation.

A typical combinatorial library is a collection of diverse compounds generated by either chemical synthesis or biological synthesis, by variably combining a number of “building blocks.”For example, a linear combinatorial library, such as a polypeptide library, is formed by combining a set of amino acid building blocks in numerous possible ways. Millions of compounds may be synthesized through such combinatorial mixing of building blocks.

Preparation and screening of combinatorial libraries is well known to those of skill in the art. Representative libraries include combinations of peptides (see e.g., U.S. Pat. No. 5,010,175; PCT Publication WO 93/20242; Furka (1991) Int. J. Pept. Prot. Res. 37:487-493; and Houghton et al. (1991) Nature 354:84-88), peptoids (PCT Publication No. WO 91/19735), random bio-oligomers (PCT Publication No. WO 92/00091), diversomers such as hydantoins, benzodiazepines and dipeptides (Hobbs et al. (1993) Proc. Nat. Acad. Sci. USA 90:6909-6913), vinylogous polypeptides (Hagihara et al. (1992) J. Amer. Chem. Soc. 114:6568), nonpeptidal peptidomimetics (Hirschmann et al. (1992) J. Amer. Chem. Soc. 114:9217-9218), small compounds (Chen et al. (1994) J. Amer. Chem. Soc. 116:2661), oligocarbamates (Cho et al., (1993) Science 261:1303), peptidyl phosphonates (Campbell et al. (1994) J. Org. Chem. 59:658), nucleic acids (Sambrook et al., 1989 Molecular Cloning—A Laboratorv Manual 2nd ed., Vol. 1-3, Cold Spring Harbor Laboratory, Cold Spring Harbor Press, N.Y.); peptide nucleic acids (see e.g., U.S. Pat. No. 5,539,083), antibodies (see e.g., Vaughn et al., (1996) Nature Biotechnology 14(3):309-314 and PCT Publication No. WO 97/00271), carbohydrates (see e.g., Liang et al., Science (1996) 274:1520-1522 and U.S. Pat. No. 5,593,853), and small organic molecule libraries (see e.g., benzodiazepines, U.S. Pat. Nos. 5,288,514 and 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337).

Devices for the preparation of combinatorial libraries are commercially available from vendors such as Advanced Chem Tech of Louisville, Ky.; 433A Applied Biosystems of Foster City, Calif.; Millipore of Bedford, Mass., etc. In addition, numerous combinatorial libraries are themselves commercially available from vendors, such as ComGenex of Princeton, N.J.; Tripos, Inc. of St. Louis, Mo.; ChemStar, Ltd. of Moscow; Martek Biosciences of Columbia, Md., etc.

### IV. Methods for Identifying Cells Expressing an HCN Channel

In another aspect of the invention, methods are provided for selecting a cell that expresses a functional HCN channel, for example, a cellular clone, that may be used for the preparation of a stable cell line. Functional HCN channels include those that are activated with membrane hyperpolarization under reduced concentrations of potassium and sodium ions, as described herein. For example, the invention provides a method of selecting a cell that expresses an HCN channel by (a) transforming a cell with a nucleic acid encoding an HCN channel; (b) incubating the cell in a saline solution containing a membrane potential sensitive dye (MPSD); (c) adding a sodium/potassium diluent to the saline solution to thereby induce hyperpolarization due to HCN channel activity; and (d) selecting a cell that is hyperpolarized upon addition of the sodium/potassium diluent. Representative saline solutions, sodium/potassium diluent solutions, and membrane potential sensitive dyes are described herein above under section III.

The cells to be tested for HCN channel activity may be any cells that are suspected to endogenously express HCN channels, including those described above. Alternatively or additionally, cells transfected with nucleic acids known or suspected to encode HCN channels may be used with the present methods. Cells may be transfected using the constructs and methods described above.

According to the disclosed methods, cells to be tested for HCN channel activity are incubated in a saline solution to which is then added a sodium/potassium diluent. The reduction in sodium and potassium ion concentration induces hyperpolarization of cells expressing a functional HCN channel. Hyperpolarization is detected by use of a membrane potential sensitive dye, to thereby allow selection of cells expressing a functional HCN channel. Suitable saline solutions, sodium/potassium diluent solutions, membrane potential sensitive dyes and methods of using the dyes to determine the membrane potential of a cell are described in section IV below.

Generally, a 30 to 50 mV change in cell membrane potential is considered to result in a good MPSD assay window. For example, a resting membrane potential of a human HCN1 channel expressing cell may be about −40 mV (see e.g., FIG. 1, which depicts cells expressing a human HCN1 channel). A cell is hyperpolarized upon addition of a sodium/potassium diluent solution as observed by a membrane potential of about −80 mV at steady state. In contrast, there may be little or no change in the membrane potential of a cell under hyperpolarizing conditions which does not express a functional HCN channel.

The hyperpolarization of a putative HCN channel expressing cell may be determined by measuring the membrane potential of the cells before the addition of the sodium/potassium diluent solution and then comparing this value to the membrane potential of the cell after the addition of the diluent solution. Alternatively, the membrane potential after the addition of the sodium/potassium diluent solution may be compared to an expected value of hyperpolarization for the particular HCN channel and cell type employed.

In some instances, a known modulator of an HCN channel may be added to the incubating saline solution or the sodium/potassium diluent solution to additionally assess the functionality of an HCN channel. For example, a cell expressing a functional HCN channel in the presence of an activating/stimulatory modulator or an inhibitory/attenuating modulator will exhibit increased or decreased magnitude of hyperpolarization, respectively, under hyperpolarizing conditions in comparison to a control magnitude of hyperpolarization. For example, representative HCN channel antagonists CsCl, ZD7288, cilobradine, ivabradine, zatebradine, capsezepine, clonidine, lidocaine, ORG 34167, and loperamide. Representative HCN channel agonists include agents that elevate intracellular cAMP concentrations, for example, dibutyryl-cAMP, 9-bromo-cAMP, forskolin, or other ligands that activate adenylate cyclase by signal transduction in the cell line employed, such as ligands for β-adrenergic receptors (adrenalin, isoproterenol, noradrenalin, etc.), or a direct agonist of an HCN channel, for example, lamotrigine.

### EXAMPLES

The following examples are included to illustrate modes of the invention. Certain aspects of the following examples are described in terms of techniques and procedures found or contemplated by the present co-inventors to work well in the practice of the invention. In light of the present disclosure and the general level of skill in the art, those of skill in the art will appreciate that the following examples are intended to be exemplary only and that numerous changes, modifications and alterations may be employed without departing from the scope of the invention.

### Example 1

### Preparation of Cells Expressing a Human HCN Channel

The expression vector pcDNA1-hHCN1, which contains human HCN1 cDNA (e.g., GenBank Accession No. NM_{—}021072); pcDNA3.1-hHCN2, which contains human HCN2 cDNA (e.g., GenBank Accession No. NM_{—}001194); and pcDNA3.1-HCN3, which contains human HCN3 cDNA (e.g., GenBank Accession No. NM_{—}020897) were used to transfect HEK-293H cells. An expression vector containing human HCN4 cDNA (e.g., GenBank Accession No. NM_{—}005447) was used to transfect CHO-K1 cells. Optionally, stable cell lines that expression human HCN channels are prepared using techniques known in the art and a selection step as described in Examples 9 and 10 herein below.

### Example 2

### Patch-Clamp Examination of HCN Channel Expressing Cells

HEK-293H cells stably transfected with human HCN1 were incubated in Hank's balanced salt solution (HBSS, Gibco catalog No. 14175-095) containing 5.3 mM KCl, 137.9 mM NaCl, 0.34 mM NaHPO₄, 4.2 mM NaHCO₃, 0.34 mM, 5.56M glucose at pH 7.4) supplemented with 1 mM CaCl₂, 1 mM MgCl₂ and 10 mM to 30 mM tetraethyl-ammonium (TEA). HCN channels were subsequently activated by the addition of a sodium/potassium diluent solution (140 mM choline, 1.0 mM CaCl₂, 1 mM MgCl₂, 10 mM HEPES, 5.5 mM glucose at pH 7.35, supplemented with 30 mM tetraethyl-ammonium) to the HBSS. The cells were examined electrophysiologically using the patch clamp technique, which is described in detail in Hamill et al. Pflugers Arch. (1981) (2): 85-100.

FIG. 1 depicts the patch-clamp (current clamp) recordings from human HCN1 channel expressing HEK-293H cells. The resting membrane potential of these cells was about −40 mV in the presence of HBSS. The membrane potential for the HCN1 current reaches about −80 mV at steady state upon addition of the low sodium/potassium diluent solution. This represents an inhibitor assay window of about 40 mV, i.e., a change in potential over which the effect of an HCN channel inhibitor may be observed. The hyperpolarization was reversed by addition of HCN channel inhibitors ZD7288 (50 μM) or CsCl (1 mM).

### Example 3

### High-Throughput Assays for Identifying HCN Channel Modulators

FIG. 2 summarizes the rationale for the HCN channel modulator assay described herein. Cells expressing an HCN channel are placed into an incubating saline solution having sodium ions at a concentration of about 140 mM and potassium ions at a concentration of about 5 mM, with a resting membrane potential of −30 to −40 mV. The HCN channel is activated when the membrane is hyperpolarized by exposing the cell to a sodium/potassium diluent solution that is added to the saline solution and results in a reduced concentration of sodium and potassium ions.

### FLIPR®³⁸⁴

HEK-293H cells stably expressing human HCN1, HCN2, or HCN3 channel were plated at a density of 45-50×10³ cells/well in a 384 well format fluorescent imaging plate reader system (FLIPR®³⁸⁴) in 50 μl/well of DMEM supplemented with 10% fetal bovine serum and penicillin/streptomycin. The cells were cultured for 24-48 hours in a 5% CO₂/air humidified incubator at 37° C.

The cells seeded on the microtiter plates were incubated in 50 μl HBSS, as described in Example 2. Optionally, HBSS was supplemented with about 25 mM of KCl to further enhance the assay window. A membrane potential sensitive dye (MPSD), Component-A of the Membrane Potential Assay Kit, (Product No. R-8034, Molecular Devices Corporation of Sunnyvale, Calif.) was dissolved in the HBSS solution (1 vial/0.5 L). The excess saline was removed to fit the residual volume of about 1 μl to 5 μl.

The HCN channels were activated by membrane hyperpolarization induced by addition of 25 μl/well of a sodium/potassium diluent solution (140 mM Choline-Cl, 1.0 mM CaCl₂, 1.0 mM MgCl₂, 10 mM HEPES, 5.5 mM glucose and 10 mM to 30 mM TEA, at pH 7.35.) MPSD (1 vial/0.5 L) was dissolved into the sodium/potassium diluent solution. TEA may be included in the HBSS saline and/or diluent solutions at this step or at subsequent steps in the assay.

Membrane potentials were measured at room temperature (21°-23°) for each of the above-described saline conditions using a fluorescent imaging plate reader system (FLIPR®, Molecular Devices of Sunnyvale, Calif.). Control cells were incubated in HBSS/MPSD without a test agent. For identification of HCN channel modulators, cells were incubated in HBSS/MPSD and the presence of a test agent as described in Examples 5 and 6 herein below.

### FLIPR®⁹⁶, FLEX⁹⁶

The above-described assay was modified for use with the FLIPR®⁹⁶ or FLEX⁹⁶ 96 well plate format. In brief, cells stably expressing HCN channels were plated at 40-80×10³ cells/plate in 100-150 μl/well of DMEM supplemented with 10% fetal bovine serum, 1× NEAA, and 1× penicillin/streptomycin. Cells were cultured for 24-48 hours in a 5% CO₂/air humidifiedincubator at 37° C. Cells were incubated in 30 μl/well of HBSS containing MPSD, as described above, for 15-45 minutes at room temperature. A single-step addition of 200 μl/well of sodium/potassium diluent solution to the HBSS resulted in a 1:7 dilution ratio of HBSS to sodium/potassium diluent solution, which resulted in sodium and potassium concentrations of 20 mM and 0.8 mM, respectively.

Membrane potentials were measured at room temperature (21°-23°) using a FLIPR® system or a FLEXSTATION® (Molecular Devices of Sunnydale, Calif.). Control cells were incubated in HBSS/MPSD without a test agent. For identification of HCN channel modulators, cells were incubated in HBSS/MPSD and the presence of a test agent as described in Examples 5 and 6 herein below.

### Example 4

### Effect of Varying Fold-Dilution of Sodium/Potassium Diluent Solution

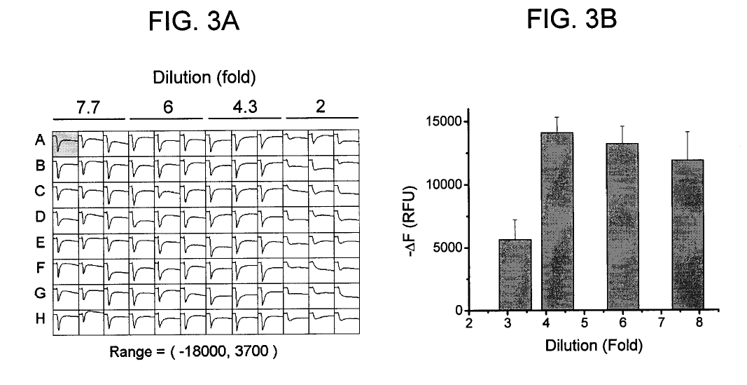

In order to further assess the effect of the cation concentration on the assay window, fluorescence responses were measured on FLIPR® from HEK-293H cells stably expressing an HCN1 channel by addition of varying ratios of sodium/potassium diluent solution to HBSS, i.e., 7.7, 6, 4.3 and 3.2 fold dilutions. FIG. 3A depicts the change in fluorescence as a function of the fold dilution of the saline solution using a FLIPR®⁹⁶ well format. FIG. 3B depicts a whole-plate screenshot of the changes in fluorescence (Relative Fluorescence Units, RFU, or counts) that reflect the respective change in membrane potential over time. The assay window (i.e., the difference in the resting membrane potential and the reversal potential following hyperpolarization) decreases as the sodium and potassium ion concentration in the incubating solution increases. In a 96-well format, a dilution greater than about 3.2 fold, such that the sodium concentration is less than about 50 mM and the potassium concentration is less than about 2 mM, result in a measurable assay window.

### Example 5

### Inhibition of Human HCN1 and HCN3 Channels as Detected using FLIPR® Assays FLIPR®³⁸⁴

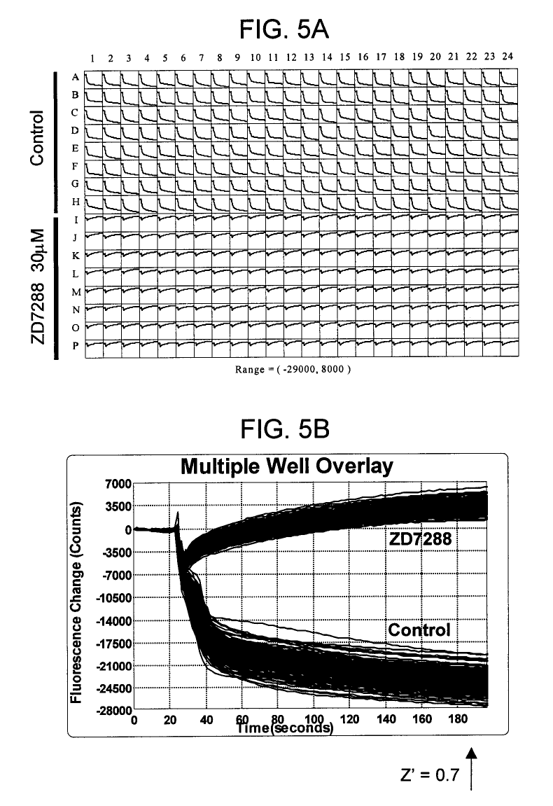

HEK-293H cells stably expressing human HCN1 channel were plated as described in Example 3 above. Cells were incubated in 50 μl HBSS supplemented MPSD with or without 30 μM of the channel inhibitor ZD7288, dissolved in <1.0% dimethyl sulfoxide (DMSO) added to the HBSS solution. After 15-45 minutes of incubation, excess solution was removed manually or with an automated liquid handling station, such as a Biotek washer, to fit the residual volume of less then3.0 μl. After 5-10 seconds of background recording using FLIPR®, HCN channels were activated by membrane hyperpolarization driven by the addition of a 25 μl sodium/potassium diluent solution as described in Example 3. The change in fluorescence (ΔF) was measured for a total assay time of about 180 seconds. As shown in FIGS. 5A-5B, presence of ZD7288 effectively blocked hyperpolarization. At 180 seconds, the average Z′ factor was 0.74.

### FLIPR®⁹⁶

HEK-293H cells stably expressing HCN1 or HCN3 channels were each plated at 40-80×10³ cells/plate in a FLIPR®⁹⁶ well format in 100-150 μl/ well of DMEM supplemented with 10% fetal bovine serum and penicillin/streptomycin. The cells were cultured for 24-48 hours in a 5% CO₂/air humidified incubatorat 37° C.

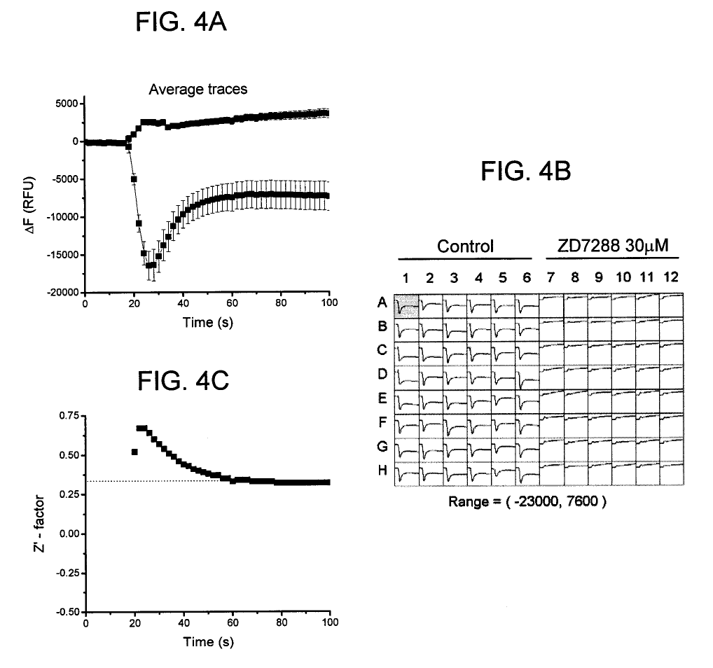

Cells were incubated in 30 μl HBSS supplemented with MPSD with or without 50 μM of the channel inhibitor ZD7288, dissolved in <1.0% dimethyl sulfoxide (DMSO) added to the HBSS solution. Cells were incubated for 15 to 45 minutes. After 5-10 seconds of background recording, the HCN channels were activated by membrane hyperpolarization driven by the addition of a 200 μl sodium/potassium diluent solution prepared as in Example 3. The change in fluorescence (ΔF) was measured for a total assay time of about 100 seconds. As shown in FIGS. 4A-4B, presence of ZD7288 effectively blocked hyperpolarization. FIG. 4C shows that the Z-factor was between about 0.25 and about 0.75 over the course of the assay. In addition, less than 0.1% false positives or false negatives were observed (covariance (CV) between 7.6 and 10.8, data not shown).

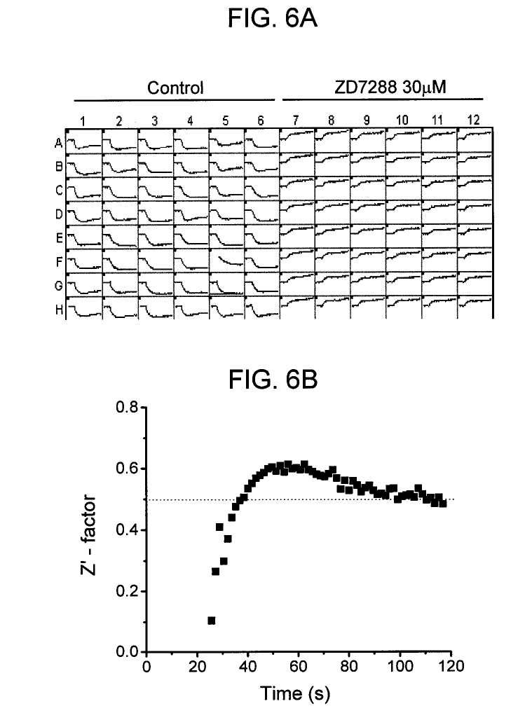

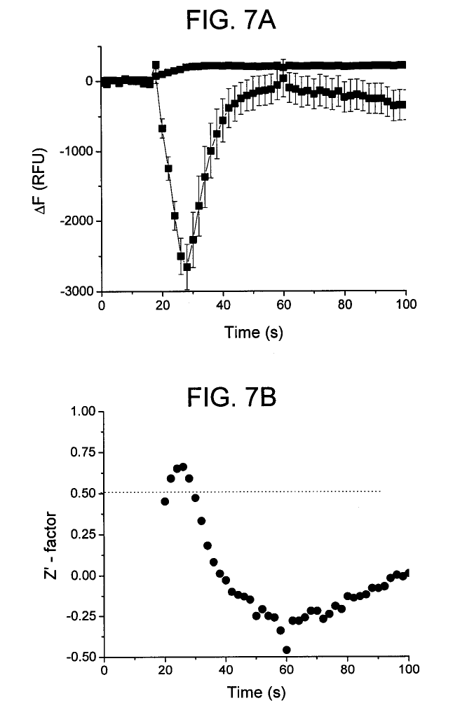

FIGS. 6A-6B shows the effect of ZD7288 on HCN3 channel expressing cells. As for HCN1 channel expressing cells, the presence of ZD7288 effectively blocked hyperpolarization. FIG. 6A depicts the whole-plate screenshot of the changes in fluorescence/membrane potential over time. FIG. 7A depicts the change in fluorescence (F) over time for control cells (upper trace) and for cells exposed to ZD7288 at a concentration of 30 μM (lower trace). FIG. 6B demonstrates the robustness of the assay. The Z-factor was between about 0.2 and 0.7 over the course of each of the assays for the HCN3 channel expressing cells. FIG. 7B shows that the Z-factor was between 0 and 0.75 at the time of hyperpolarization.

### Example 6

### Inhibition of Human HCN4 Channels as Detected using FLIPR® Assays

CHO-K1 cells stably expressing a human HCN4 channel were plated as described in Example 3 above. Using a FLIPR®⁹⁶ assay, control cells were incubated in 30 μl/well HBSS (Gibco catalog 14025-092 containing NaCl, 137.93; KCl, 5.33; KH₂PO₄, 0.44; NaHCO₃, 4.17; Na₂HPO₄, 0.34; CaCl₂, 1.26; MgCl₂, 0.49; MgSO₄, 0.41; D-Glucose 5.56). The HBSS was supplemented with MPSD (1 vial/0.5 L). The saline for test cells additionally included 30 μM of the channel inhibitor ZD7288 dissolved in <1.0% DMSO. After 15-30 minutes incubation, background fluorescence was recorded on FLIPR and the membrane hyperpolarization induced by the addition of 200 μl sodium/potassium diluent solution (140 Choline-Cl, 1.0 mM CaCl₂, 1.0 mM CaCl₂, 10 mM HEPES, 5.5 mM D-glucose, pH 7.3 with TRIS-OH), supplemented with MPSD (1 vial/0.5 L) was recorded on FLIPR.

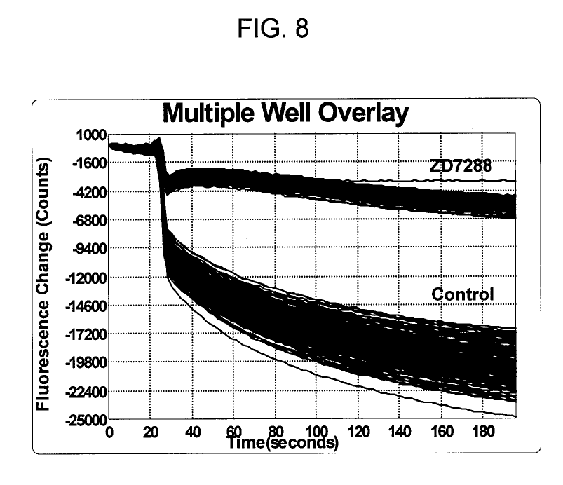

The FLIPR®³⁸⁴ well plate was prepared similarly to the FLIPR®⁹⁶ well plate except that the amounts of HBSS (optionally, HBSS was supplemented with about 25 to 50 mM of KCl to further enhance assay window) and the sodium/potassium diluent solution were adjusted to keep the dilution ratio of these solutions between about 1:10-1:30, respectively. Additionally, 50 μM ZD7288 was added to the saline containing test cells. As shown in FIG. 8, the presence of ZD7288 effectively blocked hyperpolarization of HCN4 channels in the FLIPR®³⁸⁴ assay. The Z-factor was 0.6 (FIG. 8), demonstrating that this was a robust assay. The Z′ factor correlated across plates and demonstrated a low variability (0.40 to 0.44, data not shown). In addition, less than about 0.02% false positives or false negatives were observed (covariance 11.03 and 12.05, data not shown).

### Example 7

### Effect of DMSO in HCN Channel Assays

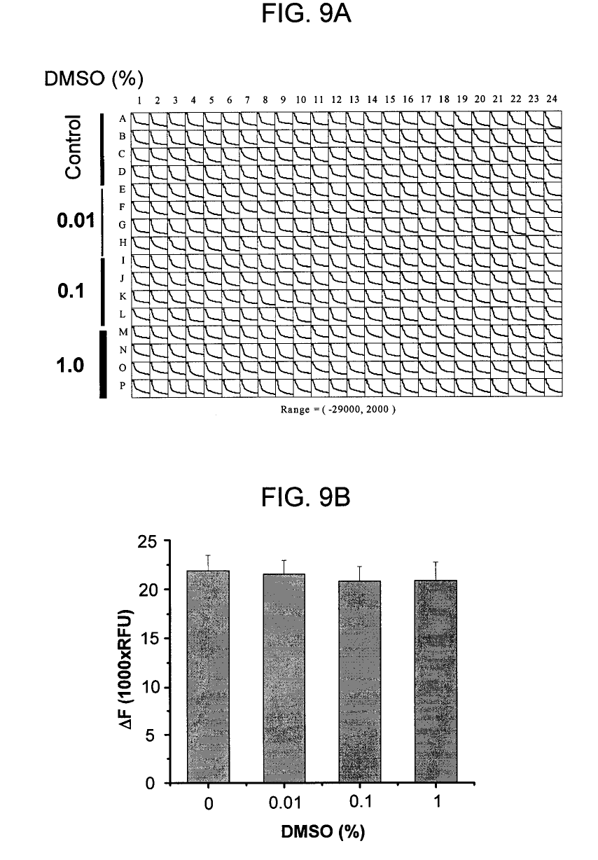

In order to assess a potential effect, if any, of DMSO in the FLIPR® assays, a FLIPR®³⁸⁴ assay was performed as described in Examples 5 with varying concentrations of DMSO. Specifically, 0% to 1% DMSO was added to the test wells containing HBSS without inhibitor. FIGS. 9A-9B demonstrate that DMSO did not significantly affect the assay results.

### Example 8

### Determination of the IC₅₀ of ZD7288 on HCN1 and HCN4 Channels

HEK-293H cells stably expressing a human HCN1 channel were plated and incubated in HBSS essentially as described in Examples 3 and 5. CHO-K1 cells stably expressing a human HCN4 channel were plated and incubated in HBSS essentially as described in Example 6. Varying concentrations of ZD7288 were added to HBSS for each of the test wells to determine the dose response of cells expressing HCN1 or HCN4 channels. The percent inhibition of hyperpolarization was calculated for a single dose using the following equation:

(1−([ΔF with ZD7288]/ΔF control]))×100.

The data were fit with a Hill equation as described by the ratio 1/(1+([test agent]/IC₅₀)^{k}, where IC₅₀ is the concentration of a test agent required to inhibit one-half of the maximum current and k is the Hill coefficient.

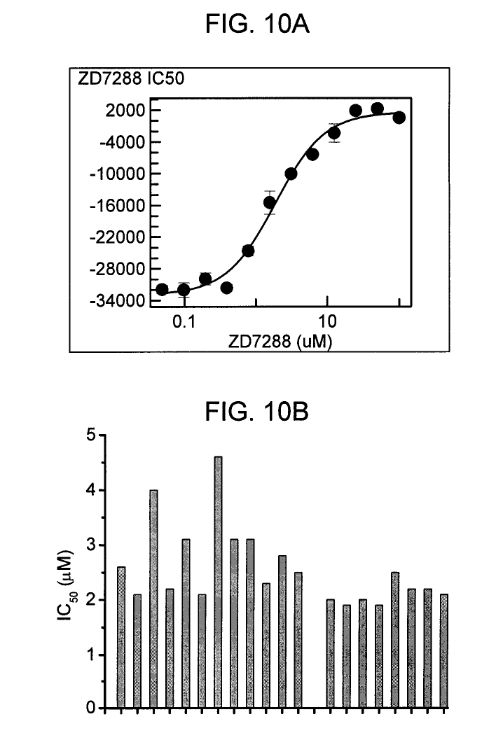

FIG. 10A depicts a typical dose response curve for a single HEK-293H HCN1 channel expressing clone in the presence of ZD7288. FIG. 10B shows the IC₅₀ values for ZD7288 obtained in a second experiment using the same HEK-293 HCN1 channel expressing clone. The IC₅₀ values for ZD7288 for group 1 ranged from 1.9 μM to 2.5 μM with a mean of 2.1 μM±0.2 μM (n=8). The range of IC₅₀ values for ZD7288 for group 2 ranged from 2.1 μM to 4.6 μM with a mean of 2.9 μM±0.8 μM (n=12). No statistical difference was found between the two means.

Similarly, ZD7288 IC₅₀ values on HCN4 channel were determined in a set of two experiments. The IC₅₀ values for ZD7288 for group 1 ranged from 5.77 μM to 10.57 μM with a mean of 9.07 μM±2.31 μM (n=8). The IC₅₀ values for ZD7288 for group 2 ranged from 6.56 μM to 12.33 μM with a mean of 9.06 μM±2.61 μM (n=8) (data not shown). No statistical difference was found between the two experiments.

### Example 9

### Selection of Cell Lines Expressing HCN1 or HCN3 Channels

HEK-293H cells were transfected with constructs encoding a human HCN1 channel (pcDNA3.1-HCN1) or a human HCN3 channel (pcDNA3.1-HCN3), essentially as described in Example 1. Cell lines were generated from a single clone according to standard protocols. The cells were plated and incubated in HBSS/MPSD (control cells) or HBSS/MPSD plus 50 μM ZD7288 (test cells) using a 96 well plate format, followed by the addition of the sodium/potassium diluent solution using a FLEX⁹⁶ assay as described in Examples 3 and 5. Cells that stably express functional HCN channels were selected based upon an ability to hyperpolarize upon the addition of sodium/potassium diluent, which is blocked by the presence of ZD7288.

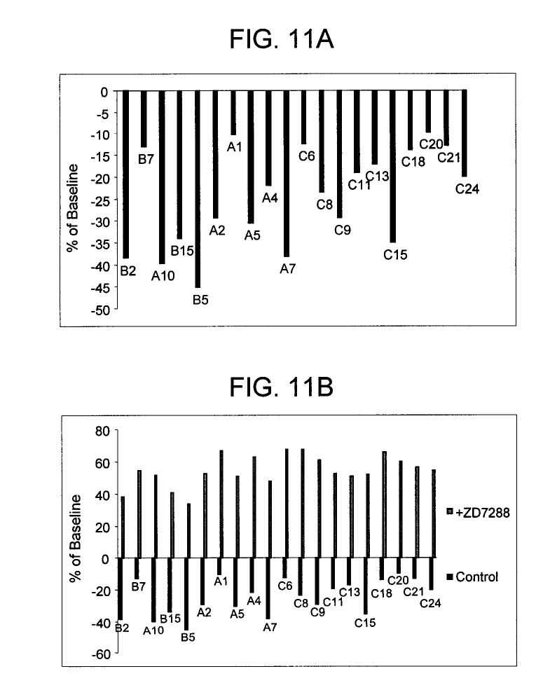

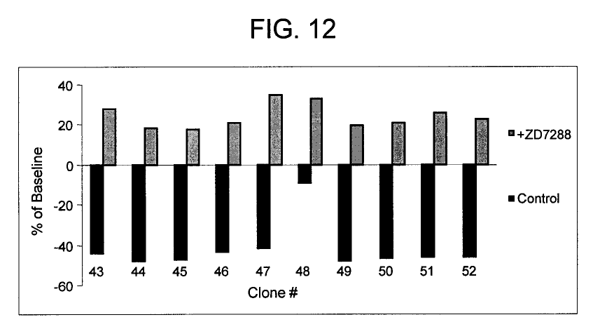

FIGS. 11A-11B and FIG. 12 depict the percentage of MPSD fluorescence change (% baseline) with (FIG. 11B, FIG. 12) or without (FIG. 11A) addition of ZD7288 for representative clones expressing HCN3 (FIGS. 11A-11B) or HCN1 (FIG. 12) channels. A large % baseline value corresponds to a greater magnitude of hyperpolarization of the cell following addition of sodium/potassium diluent.

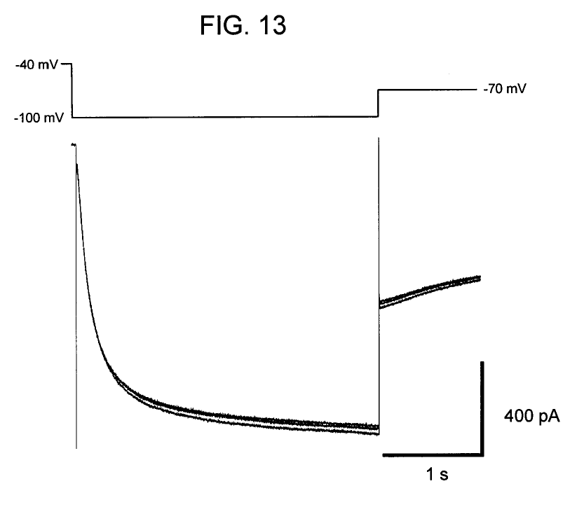

A stable cell line was selected as expressing a functional HCN1 channel using the FLEX⁹⁶ assay, and patch-clamping was used to confirm the electrophysiological properties of representative cells. The cells were clamped to a holding potential of −40 mV. Currents were elicited every 30 seconds by hyperpolarizing pulses from a holding voltage to −100 mV followed by a step to −70 mV (FIG. 13)

### Example 10

### Selection of Stable Cell Lines Expressing HCN2 Channels

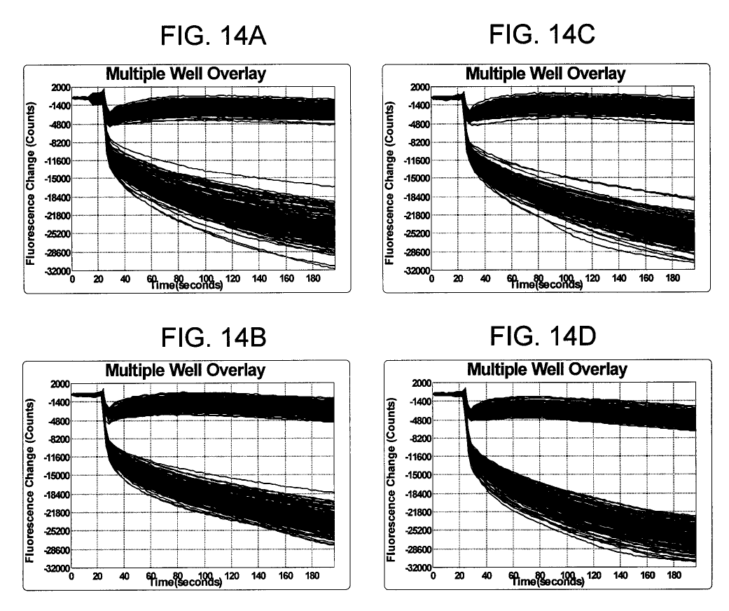

HEK-293H cells were transfected with constructs encoding a human HCN2 channel essentially as described in Example 1. Cell lines were generated from a single clone according to standard protocols. The cells were incubated in HBSS/MPSD (control cells) or HBSS/MPSD and 30 μM ZD7288 (test cells) using a 384 well plate format, followed by the addition of the sodium/potassium diluent solution essentially as described in Examples 3 and 5. FIGS. 14A-14D show the effect of ZD7288 on each of the four clones expressing HCN2 channels. FIGS. 14A (clone #4), 14B (clone #8), 14C (clone #11), and 14D (clone #12) each depict the change in fluorescence over time for cells incubated in saline only (overlaid lower traces ) and for cells incubated in saline containing the HCN channel inhibitor ZD7288 (overlaid upper traces ). The presence of ZD7288 effectively blocked hyperpolarization for each of the clones. The clones showed a similar magnitude of hyperpolarization with a mean signal between −26368 and −23600 relative fluorescence units (RFU, Table 1). The Z-factor was between 0.56 and 0.64, and the covariance (CV) was between from 6.6 to 9.9. Clones #8 and #12 showed the greatest HCN channel-induced response as determined by the difference in RFU in control versus RFU in the presence of ZD7288. See Table 1.

TABLE 1
Effect of ZD7288 on Cells Stably Expressing HCN2 Channels

| | Clone #4 | | Clone #8 | | Clone #11 | | Clone #12 | |
|---|---|---|---|---|---|---|---|---|
| | | Standard | | Standard | | Standard | | Standard |
| | Mean | Deviation | Mean | Deviation | Mean | Deviation | Mean | Deviation |
| Δfluorescence | −24421 | 2390 | −23600 | 1724 | −25477 | 1877 | −26367 | 1714 |
| (saline only) | | | | | | | | |
| Δfluorescence | −2392 | 864 | −3006 | 912 | −2413 | 842 | −4834 | 898 |
| (saline + ZD7288) | | | | | | | | |
| CV | 9.8 | | 7.3 | | 7.4 | | 6.5 | |
| Z-factor | 0.56 | | 0.62 | | 0.65 | | 0.64 | |

What is claimed:

1. A method for identifying a modulator of a hyperpolarization-activated cyclic nucleotide gated (HCN) channel comprising the steps of:
(a) providing a cell expressing an HCN channel in a saline solution comprising a membrane potential sensitive dye;
(b) contacting the cell with a test agent;
(c) adding a sodium/potassium diluent to the saline solution of (a) to thereby induce hyperpolarization, wherein the diluent is optionally supplemented with the membrane potential sensitive dye; and
(d) assaying attenuation or enhancement of hyperpolarization, as compared to a control level of hyperpolarization, wherein attenuation of hyperpolarization indicates that the test agent is an HCN channel antagonist, and wherein enhancement of hyperpolarization indicates that the test agent is an HCN channel agonist.

2. The method of claim 1, wherein the HCN channel comprises alpha subunits selected from the group consisting of HCN1, HCN2, HCN3, and HCN4 alpha subunits.

3. The method of claim 1, wherein the HCN channel is a heteromeric channel.

4. The method of claim 1, wherein the HCN channel is a homomeric channel.

5. The method of claim 2, wherein the HCN channel comprises an HCN1 alpha subunit and an HCN2 alpha subunit.

6. The method of claim 2, wherein the HCN channel comprises an HCN1 alpha subunit and an HCN3 alpha subunit.

7. The method of claim 2, wherein the HCN channel comprises an HCN1 alpha subunit and an HCN4 alpha subunit.

8. The method of claim 2, wherein the HCN channel comprises an HCN2 alpha subunit and an HCN3 alpha subunit.

9. The method of claim 2, wherein the HCN channel comprises an HCN2 alpha subunit and an HCN4 alpha subunit.

10. The method of claim 2, wherein the HCN channel comprises an HCN3 alpha subunit and an HCN4 alpha subunit.

11. The method of claim 1, wherein the HCN channel comprises HCN alpha subunits and an auxiliary beta subunit.

12. The method of claim 11, wherein the auxiliary beta subunit is encoded by a member of the KCNE gene family.

13. The method of claim 11, wherein the alpha subunits and the auxiliary beta subunit are expressed from a single vector.

14. The method of claim 11, wherein the alpha subunits and the auxiliary beta subunit are expressed from different vectors.

15. The method of claim 1, wherein the cell expressing an HCN channel endogenously express an HCN channel.

16. The method of claim 15, wherein the cell expressing an HCN channel is selected from the group consisting of cardiac cells, neural cells, photoreceptor cells, and taste bud cells.

17. The method of claim 16, wherein the cardiac cells are selected from the group consisting of sino-atrial node cells, atrial muscle cells, and ventricular muscle cells.

18. The method of claim 16, wherein the neural cells are selected from the group consisting of olfactory cells, cerebral cortical cells, hippocampal cells, thalamus cells, amygdale cells, superior collicular cells, inferior collicular cells, cerebellar cells, Purkinje cells, and neural stem cells.

19. The method of claim 1 wherein the cell stably expresses the HCN channel.

20. The method of claim 1, wherein the cell transiently expresses the HCN channel.

21. The method of claim 1, wherein the cell is a eukaryotic cell.

22. The method of claim 21, wherein the eukaryotic cell is selected from the group consisting of amphibian cells, yeast cells, and mammalian cells.

23. The method of claim 22, wherein the eukaryotic cell is a mammalian cell.

24. The method of claim 23, wherein the mammalian cell is selected from the group consisting of COS cells, mouse L cells, CHO cells, human embryonic kidney cells, and African green monkey cells.

25. The method of claim 1, wherein the saline solution of step (a) comprises at least 50 mM of sodium ions and at least 5 mM of potassium ions.

26. The method of claim 1, wherein the saline solution of step (a) is selected from the group consisting of Ringer's Lactate, Tyrode's buffer, Kreb's buffer, Hank's balanced salt solution, and modified Hank's balanced salt solution.

27. The method of claim 1 wherein the sodium/potassium diluent reduces the cation concentration of the saline solution by at least five fold.

28. The method of claim 1 wherein the sodium/potassium diluent reduces the cation concentration of the saline solution by at least ten fold.

29. The method of claim 1 wherein the sodium/potassium diluent reduces the cation concentration of the saline solution by at least twenty fold.

30. The method of claim 1 wherein the sodium/potassium diluent reduces the cation concentration of the saline solution by at least thirty fold.

31. The method of claim 1, wherein the sodium/potassium diluent reduces the cation concentration of the saline solution by at least fifty fold.

32. The method of claim 1, wherein the sodium/potassium diluent reduces the concentration of sodium ions in the saline solution to less than about 30 mM.

33. The method of claim 1, wherein the sodium/potassium diluent reduces the concentration of potassium ions in the saline solution to less than about 10 mM.

34. The method of claim 1, wherein the saline solution further comprises an impermeant cation.

35. The method of claim 34, wherein the impermeant cation is selected from the group consisting of N-methyl-D-glucamine, choline, tetraethylammonium, tetrethymethyammonium, and tetrapropylammonium.

36. The method of claim 1, wherein the sodium/potassium diluent further comprises an HCN channel impermeant cation.

37. The method of claim 36, wherein the impermeant cation is selected from the group consisting of N-methyl-D-glucamine, choline, tetraethylammonium, tetrethymethyammonium, and tetrapropylammonium.

38. The method of claim 1, wherein the membrane potential sensitive dye is selected from the group consisting of oxonols, carbocyanine, rhodamines, and derivatives thereof.

39. The method of claim 38, wherein the membrane potential sensitive dye is an oxonol derivative.

40. The method of claim 39, wherein the oxonol derivative is a 3-bis-barbituric acid oxonol derivative.

41. The method of claim 40, wherein the 3-bis-barbituric acid oxonol derivative is bis-(1,3-dibutylbarbituric acid) trimethine oxonol [DiBac₄(3)], bis-(1,3-diethylthiobarbituric acid)trimethine oxonol, bis-(1,3-dibutylbarbituric acid)pentamethine oxonol, or a combination thereof.

42. The method of claim 1, wherein the membrane potential sensitive dye is suitable for use in a fluorescent imaging plate reader system.

43. The method of claim 1, which is performed on a high-throughput scale.

44. The method of claim 1, wherein the modulator is an agent that blocks an HCN channel, an agent that inhibits an HCN channel, an agent that activates an HCN channel, or an agent that enhances HCN channel conductance.

45. The method of claim 1, wherein the test agent is selected from the group consisting of small molecules, antibodies or fragments thereof, peptides, proteins, oligopeptides polysaccharides, lipids, fatty acids, nucleic acids, and nucleic acid-protein fusions.

46. A method of selecting a cell line that expresses a hyperpolarization-activated cyclic nucleotide gated (HCN) channel comprising the steps of:
(a) providing a cell suspected to express a hyperpolarization-activated cyclic nucleotide gated (HCN) channel in a saline solution supplemented with a membrane potential sensitive dye (MPSD);
(b) adding a sodium/potassium diluent solution to the saline solution of (a) to thereby induce hyperpolarization; and
(c) selecting a cell that is hyperpolarized upon addition of the sodium/potassium diluent solution.

47. The method of claim 46, further comprising:
(d) selecting a cell that shows inhibition of induced hyperpolarization when contacted with an HCN channel inhibitor.

48. The method of claim 47, wherein the HCN channel inhibitor is CsCl, ZD7288, ORG 34167, cilobradine, ivabradine, zatebradine, capsezepine, lidocaine, or loperamide.

49. The method of claim 46, further comprising:
(d) selecting a cell that shows enhancement of induced hyperpolarization when contacted with an HCN channel activator.

50. The method of claim 49, wherein the HCN channel activator induces elevated levels of cAMP.

51. The method of claim 46, further comprising:
(d) establishing a stable cell line using the cell of step (c).

52. A cell line prepared according to the method of claim 46.
